(19) United States
(12) Patent Application Publication
(10) Pub. No.: US 2025/0137908 A1
(43) Pub. Date: May 1, 2025

(54) SPERM SORTING DEVICE

(71) Applicant: National Yang-Ming Chiao Tung University, Hsinchu (TW)

(72) Inventors: Bor-Ran Li, Hsinchu (TW); Siao-Yun Peng, New Taipei (TW); Chung-Hsien Huang, Tainan (TW)

(21) Appl. No.: 18/444,744

(22) Filed: Feb. 18, 2024

(30) Foreign Application Priority Data
Oct. 25, 2023 (TW) 112140750

(51) Int. Cl. G01N 15/149

(52) U.S. Cl. CPC G01N 15/149

(57) ABSTRACT

A sperm sorting device includes a foundation unit and a channel unit. The foundation unit includes a foundation wall, a surrounding wall extending from a periphery of the foundation wall, and a plurality of column portions. The channel unit has an inner bordering wall, a base plate surrounded by and connected to the inner bordering wall and having first and second surfaces, and a plurality of channel holes. The column portions of the foundation unit respectively extend through the channel holes. A distance between a summit surface of each of the column portions and the foundation wall is not smaller than a distance between the second surface and the foundation wall. Each of the channel holes has a selection space that is not occupied by the respective one of the column portions and that gradually reduces in size in a direction from the foundation wall to the base plate.

### CROSS-REFERENCE TO RELATED APPLICATION

This application claims priority to Taiwanese Invention Patent Application No. 112140750, filed on Oct. 25, 2023, the entire disclosure of which is incorporated by reference herein.

### FIELD

The disclosure relates to a sorting device, and more particularly to a sperm sorting device for increasing sperm recovery rate.

### BACKGROUND

Assisted reproductive methods have been receiving more attention as infertility rates have been on the rise over the years. No matter which assisted reproductive method is used, and whether it's in vitro fertilization (IVF) or intra-cytoplasmic sperm injection (ICSI), sperms are required to be sorted beforehand in order to increase the percentage of motile sperms in semen used in the reproductive process.

Among conventional sperm sorting methods, the swim-up method and the density gradient centrifugation are commonly used. However, procedure time of the swim-up method is long, which causes motility of the sperms to decrease, thereby lowering sperm recovery rate. The density gradient centrifugation produces reactive oxygen species (ROS) in the process, which may damage the DNA of the sperms, and result in the sorted sperms having an increased fertilization failure rate or impaired embryo development. Hence, increasing sperm recovery rate while simultaneously preventing sperms from being damaged during the sorting process is a problem that needs to be mitigated in relevant fields.

### SUMMARY

Therefore, an object of the disclosure is to provide a sperm sorting device that can alleviate at least one of the drawbacks of the prior art.

According to the disclosure, the sperm sorting device includes a foundation unit and a channel unit. The foundation unit includes a foundation wall, a surrounding wall, and a plurality of column portions. The surrounding wall extends from a periphery of the foundation wall, and cooperates with the foundation wall to define an accommodation space. The plurality of column portions extend from the foundation wall into the accommodation space, and each of the column portions has a summit surface on a distal end thereof. The channel unit is disposed in the accommodation space, and has an inner bordering wall, a base plate, and a plurality of channel holes. The inner bordering wall is disposed on the foundation wall. The base plate is surrounded by and connected to the inner bordering wall, and has a first surface and a second surface that are opposite to each other and in that are respectively proximal to and distal from the foundation wall. The plurality of channel holes extend through the first surface and the second surface of the base plate. The column portions of the foundation unit respectively extend through the channel holes. A distance between the summit surface of each of the column portions and the foundation wall is not smaller than a distance between the second 10 surface of the base plate and the foundation wall. Each of the channel holes has a selection space that is not occupied by the respective one of the column portions and that gradually reduces in size in a direction from the foundation wall to the base plate.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is an exploded perspective view illustrating an embodiment of a sperm sorting device according to the disclosure.

FIG. 2 is a sectional side view of the embodiment.

FIG. 3 is a schematic top view of the embodiment.

FIG. 4 is an enlarged fragmentary sectional view of the embodiment illustrating a selection space.

FIG. 5 is an enlarged fragmentary sectional view of another embodiment according to the disclosure.

FIG. 6 illustrates a graph of an unsorted semen solution and a graph of a sorted semen solution, which respectively show a relationship between linearity and straight line velocity (VSL) of sperms in the unsorted semen solution and a relationship between linearity and straight line velocity (VSL) of sperms in the sorted semen solution.

FIG. 7 illustrates a graph of the unsorted semen solution, a graph of the sorted semen solution, and a graph of a residual semen solution, which respectively show the straight line velocity (VSL), the average path velocity (VAP), and the curvilinear velocity (VCL) of the sperms therein.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

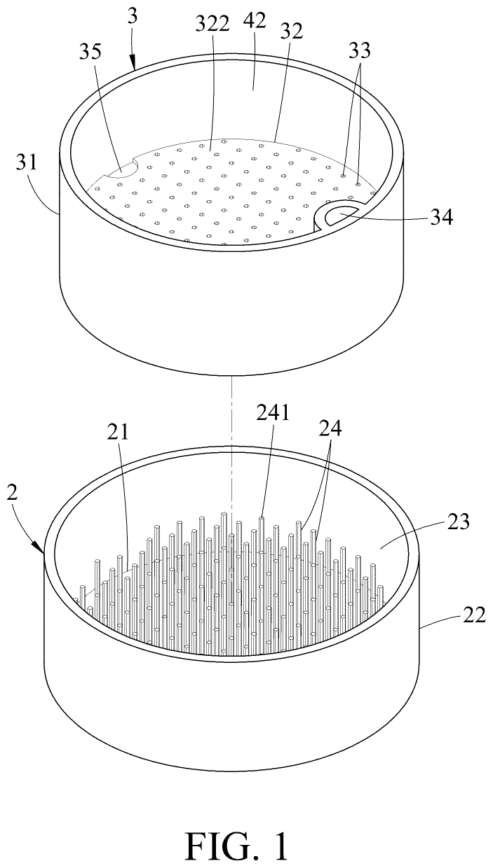

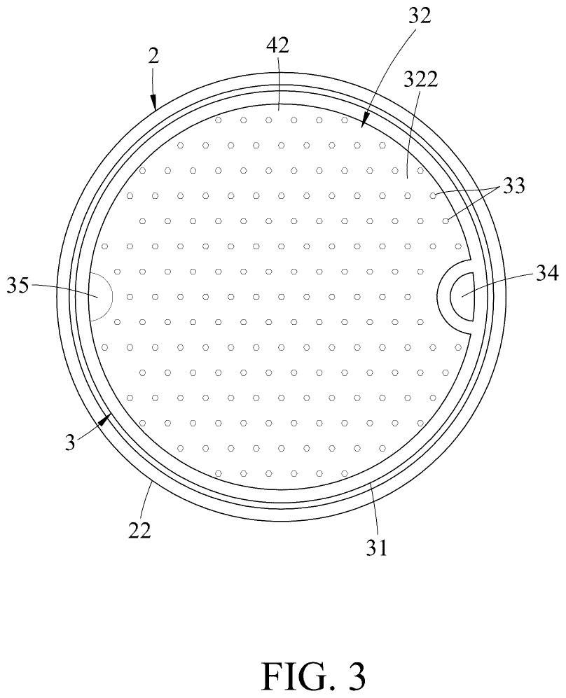

Referring to FIGS. 1 to 3, an embodiment of a sperm sorting device according to the disclosure is adapted for sorting sperms in a semen solution, increasing the percentage of motile sperms therein. The sperm sorting device includes a foundation unit 2 and a channel unit 3. The foundation unit 2 and the channel unit 3 are made of plastic (for example, acrylic). In this embodiment, the foundation unit 2 and the channel unit 3 may be made of biocompatible UV-curing resin, for example, CURO Splint, Somos BioClear, and HT-EEM324, thereby decreasing a mortality rate of sperms during sorting.

The foundation unit 2 includes a foundation wall 21, a surrounding wall 22 extending from a periphery of the foundation wall 21, and cooperating with the foundation wall 21 to define an accommodation space 23, and a plurality of column portions 24 extending from the foundation wall 21 into the accommodation space 23, and spaced apart from each other. Each of the column portions 24 has a summit surface 241 on a distal end thereof.

The channel unit 3 is disposed in the accommodation space 23, and has an inner bordering wall 31 disposed on the foundation wall 21, a base plate 32 surrounded by and connected to the inner bordering wall 31, a plurality of channel holes 33, an inlet 34 extending through the base plate 32, and a recess 35. The base plate 32 has a first surface 321 and a second surface 322 that are opposite to each other and that are respectively proximal to and distal from the foundation wall 21. The channel holes 33 extend through the first surface 321 and the second surface 322 of the base plate 32. The recess 35 extends from the second surface 322 of the base plate 32 towards the first surface 321. The first surface 321 of the base plate 32, the inner bordering wall 31, and the foundation wall 21 cooperatively define a solution-carrying space 41 thereamong, and the solution-carrying space 41 is adapted for containing a semen solution.

The second surface 322 of the base plate 32 and the inner bordering wall 31 cooperatively define a sample space 42 adapted to contain a culture medium. The inlet 34 extends along the inner bordering wall 31, and extends through the first surface 321 and the second surface 322. The channel holes 33 and the inlet 34 are in spatial communication with the solution-carrying space 41. The inlet 34 has an end opposite to the foundation wall 21, and a distance between the end of the inlet 34 and the foundation wall 21 is greater than a distance between the second surface 322 of the base plate 32 and the foundation wall 21, so that the semen solution in the culture medium is prevented from erroneously entering the solution-carrying space 41 through the inlet 34. The semen solution in the solution-carrying space 41 passes through the channel holes 33, and flows into the culture medium in the sample space 42 as sorted semen solution. A portion of the sorted semen solution flows into the recess 35, and may be collected by the user therefrom.

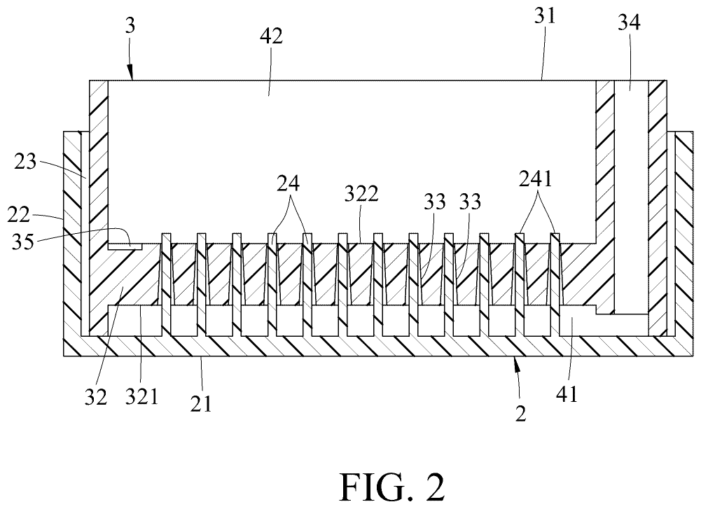

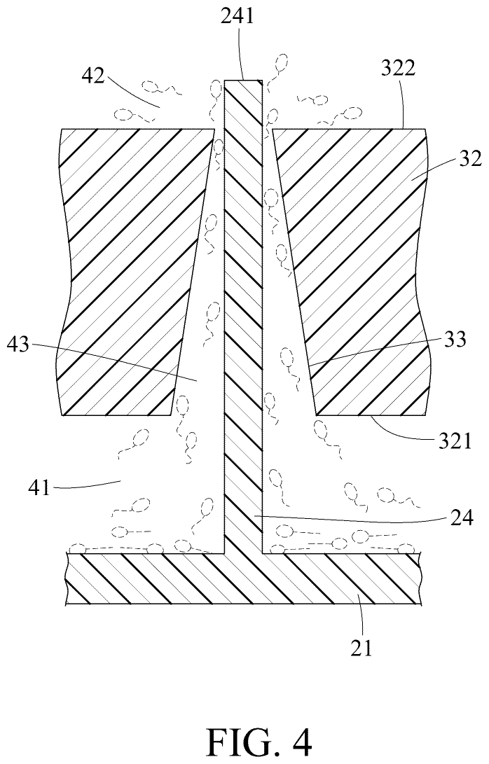

Referring to FIGS. 2 and 4, the column portions 24 of the foundation unit 2 respectively extend through the channel holes 33. A distance between the summit surface 241 of each of the column portions 24 and the foundation wall 21 is not smaller than a distance between the second surface 322 of the base plate 32 and the foundation wall 21. Each of the channel holes 33 has a selection space 43 that is not occupied by the respective one of the column portions 24 and that gradually reduces in size in a direction from the foundation wall 21 to the base plate 32. During the process of the swim up method, the sperms in the semen solution are inclined to swim from the semen solution to the culture medium. Compared to a selection space with a uniform size, a design of each of the selection spaces 43 further allows the sperms to move towards an end of the selection space 43 with a smaller size, so that the sperms swim from the semen solution to the culture solution more smoothly, and sperm recovery rate increases.

Each of the channel holes 33 is a polygonal hole, and each of the column portions 24 has a polygonal cross section. In other embodiments, combinations of the channel holes 33 and the column portions 24 may be, for example, each of the channel holes 33 is a polygonal hole, and each of the column portions 24 has a non-polygonal cross section, or each of the channel holes 33 is a non-polygonal hole, and each of the column portions 24 has a polygonal cross-section. Sperms have a boundary following behavior, and are inclined to follow a boundary of each of the channel holes 33 and/or a side surface of each of the column portions 24 to swim into the sample space 42. In an embodiment, when each of the channel holes 33 is a polygonal hole and/or when each of the column portions 24 has a polygonal cross-section, amounts of the channel holes 33 and the column portions 24 that are respectively disposed on the base plate 32 and the foundation wall 21 increase, which in turn increases an amount of the selection spaces 43, thereby increasing a total amount of boundary areas of the channel holes 33 and/or side surface areas of the column portions 24, and allowing the sperms to come into contact with the boundaries of the channel holes 33 and the side surfaces of the column portions 24 so as to have a greater chance to follow the boundaries and the side surfaces to the sample space 42. Compared to the embodiment, another embodiment, in which when each of the channel holes 33 is not a polygonal hole and/or when each of the column portions 24 does not have a polygonal cross-section, the total amount of the boundary areas of the channel holes 33 and/or the side surface areas of the column portions 24 may not be as many as those of the embodiment, may not allow the sperms to have the same great chance to follow the boundaries and the side surfaces to the sample space 42. Hence, through the polygonal shape of each of the channel holes 33 and/or the shape of each of the column portions 24, the sperm recovery rate is increased.

It should be noted that, a shape of each of the selection spaces 43 is determined by a shape of each of the channel holes 33 and a shape of each of the column portions 24. In this embodiment, each of the column portions 24 has a uniform cross-section in the direction from the foundation wall 21 to the base plate 32, and each of the channel holes 33 gradually shrinks in size in the direction from the foundation wall 21 to the base plate 32. Each of the column portions 24 has a hexagonal cross-section, and each of the channel holes 33 is a hexagonal hole. The column portions 24 and the channel holes 33 are respectively distributed on the foundation wall 21 and the base plate 32 to form a close-packed array. As such, amounts of the channel holes 33 and the column portions 24 that are respectively disposed on said base plate 32 and the foundation wall 21 increase, and an amount of the selection spaces 43 increases, thereby increasing the sperm recovery rate.

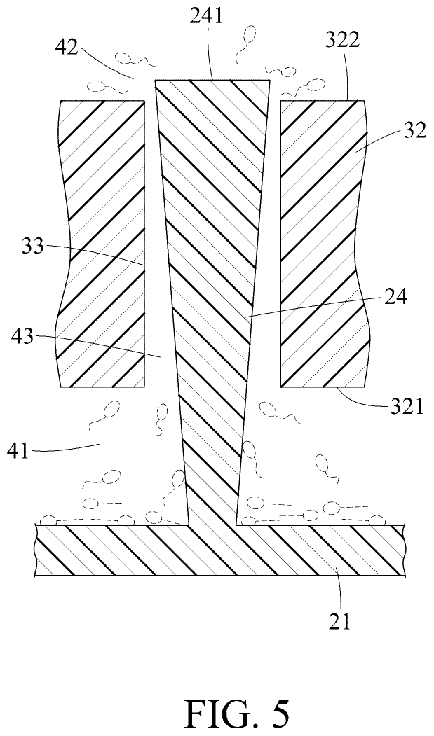

Referring to FIG. 5, another embodiment is similar to the embodiment in FIG. 1, but each of the column portions 24 has a cross-section gradually growing in size in the direction from the foundation wall 21 to the base plate 32, and each of the channel holes 33 has uniform shape and size in the direction from the foundation wall 21 to the base plate 32.

It should be noted that the shape of the cross-section of each of the column portions 24, and/or each of the channel holes 33 may be changed as necessary. For example, in other embodiments, each of the column portions 24 may have a circular cross-section, and each of the channel holes 33 may be a circular hole.

In other embodiments, the selection spaces 43 may be adjusted by altering a thickness of the base plate 32 and/or the distance between the base plate 32 and the foundation wall 21. A volume of the solution-carrying space 41 may also be adjusted in the same way.

It should be noted that, in this embodiment, a position of the foundation unit 2 is under that of the channel unit 3, but in other embodiments, the relative positons of the foundation unit 2 and the channel unit 3 may be different, for example, the foundation unit 2 may be disposed above the channel unit 3; however, the direction of the gradual reduction of size of the selection spaces 43 must be the direction from the foundation wall 21 to the base plate 32.

Compared to the conventional sperm sorting methods such as the density gradient centrifugation, which may damage sperms during the sorting process, and the swim up method, which uses sorting channels with holes having uniform widths, and may lower sperm motility after a long process time, the sperm sorting device of the present disclosure prevents sperms from being damaged during the sorting process, and the shape of the selection spaces 43 increases the sperm recovery rate and reduces time for the sorting process, thereby alleviating the problem of sperm losing motility due to long sorting process time.

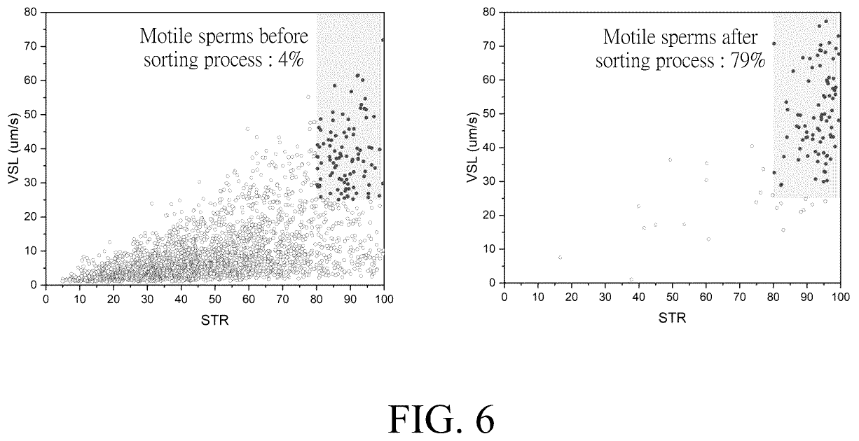

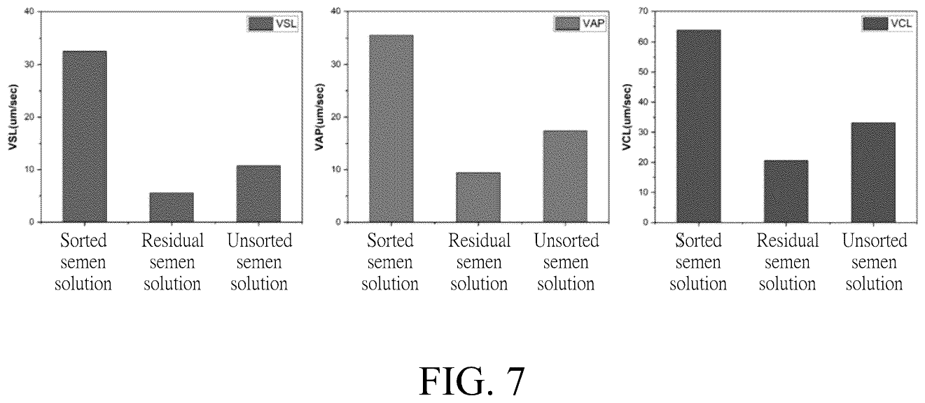

Referring to FIGS. 6 and 7, an example of a sperm sorting process implemented by the sperm sorting device and a test result analysis of a sperm solution before and after the sorting process are as follows.

The test results of the semen solution are obtained by photographing the semen solution before and after the sorting process using an optical microscope. The test results are used for observing activity of the sperms in the semen solution, and a straight line velocity (VSL), an average path velocity (VAP), and a curvilinear velocity (VCL) of the sperms in the semen solution are calculated according to the observation of the test results.

Specifically, before the sorting process, the semen solution is photographed by the optical microscope to obtain an unsorted semen solution image data. A user pours the semen solution into the solution-carrying space 41 via the inlet 34, and disposes a culture medium in the sample space 42, so that a 30-minute sorting process begins. The culture medium is a modified human tubal fluid medium (mHTF, company: LifeGlobal; specifications: CE Marked (CE 0086)).

During the sorting process, the sperms, following the swim against flow behavior and the boundary following behavior, swim along the side surfaces of the column portions 24 and/or the boundaries of the channel holes 33 through the selection spaces 43, and into the culture medium in the selection space 42, and a portion of the sperms in the culture medium flow into the recess 35. Sperms with insufficient motility do not have enough energy to swim to the culture medium, and remain in the solution-carrying space 41. After the sorting process is over, a sorted semen solution and a residual semen solution are respectively obtained by collecting a mixture of sperms and culture medium in the recess 35 and a mixture of sperms and semen solution that remain in the solution-carrying space 41, and the sorted semen solution and the residual semen solution are observed under the optical microscope to obtain a sorted semen solution image data and a residual semen solution image data. A computer assisted semen analysis (CASA) apparatus is used to analyze the unsorted semen solution image data, the sorted semen solution image data, and the residual semen solution image data, thereby obtaining the straight line velocity (VSL), the average path velocity (VAP), and the curvilinear velocity (VCL) of the sperms of the respective image data. Results of the analysis are then recorded as shown in FIGS. 6 and 7.

Referring to FIG. 6, FIG. 6 shows a graph of the unsorted semen solution and a graph of the sorted semen solution, which respectively illustrate a relationship between linearity and straight line velocity (VSL) of sperms in the unsorted semen solution and a relationship between linearity and straight line velocity (VSL) of sperms in the sorted semen solution. The solid circles represent motile sperms that meet a high motility standard, with a linear velocity of over 25 u m/s and a straightness (STR) of over 80. The hollow circles represent sperms that do not meet the aforementioned high motility standard (i.e., sperms with lower motility). Straightness (STR) is calculated by a ratio of straight line velocity (VSL) over average path velocity (VAP). A percentage of sperms meeting the high motility standard is 4% before the sperm sorting process and is raised to 79% after the sperm sorting process using the sperm sorting device.

Referring to FIG. 7, FIG. 7 illustrates a graph of the unsorted semen solution, a graph of the sorted semen solution, and a graph of the residual semen solution, which respectively show the straight line velocity (VSL), the average path velocity (VAP), and the curvilinear velocity (VCL) of the sperms thereof. The sperms from the residual semen solution are unable to swim into the sample space 42 due to lack of motility, therefore the straight line velocity (VSL), the average path velocity (VAP), and the curvilinear velocity (VCL) thereof are lower in comparison to those of the sperms from the unsorted semen solution. The motility of the sperms from the sorted semen solution is higher, therefore the straight line velocity (VSL), the average path velocity (VAP), and the curvilinear velocity (VCL) thereof are significantly increased. Hence, the sperm sorting device may achieve the objective of sorting sperms, so as to acquire the sorted semen solution with sperms having high motility.

In conclusion, the structural features of each of the selection spaces 43 gradually reducing in size in the direction from the foundation wall 21 to the base plate 32, each of the channel holes 33 being a hexagonal hole, and each of the column portions 24 having a hexagonal cross-section, facilitate the sperms to swim into the culture medium in the sample space 42, prevent the sperms from being damaged during the sperm sorting process, and reduce the time needed and motility loss of the sperms in the sperm sorting process, thereby achieving the objective of the disclosure.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A sperm sorting device comprising:
a foundation unit that includes
a foundation wall,
a surrounding wall extending from a periphery of said foundation wall, and cooperating with said foundation wall to define an accommodation space, and
a plurality of column portions extending from said foundation wall into said accommodation space, each of said column portions having a summit surface on a distal end thereof; and
a channel unit that is disposed in said accommodation space, and that has
an inner bordering wall disposed on said foundation wall,
a base plate surrounded by and connected to said inner bordering wall, said base plate having a first surface and a second surface that are opposite to each other and that are respectively proximal to and distal from said foundation wall, and
a plurality of channel holes extending through said first surface and said second surface of said base plate;
wherein said column portions of said foundation unit respectively extend through said channel holes, a distance between said summit surface of each of said column portions and said foundation wall being not smaller than a distance between said second surface of said base plate and said foundation wall, each of said channel holes having a selection space that is not occupied by the respective one of said column portions and that gradually reduces in size in a direction from said foundation wall to said base plate.

2. The sperm sorting device as claimed in claim 1, wherein each of said channel holes is a polygonal hole.

3. The sperm sorting device as claimed in claim 1, wherein each of said column portions has a polygonal cross-section.

4. The sperm sorting device as claimed in claim 1, wherein:
each of said channel holes is a hexagonal hole; and
each of said column portions has a hexagonal cross-section.

5. The sperm sorting device as claimed in claim 1, wherein:
each of said column portions has a uniform cross-section in the direction from said foundation wall to said base plate; and
each of said channel holes gradually shrinks in size in the direction from said foundation wall to said base plate.

6. The sperm sorting device as claimed in claim 1, wherein:
each of said column portions has a cross-section gradually growing in size in the direction from said foundation wall to said base plate; and
each of said channel holes has uniform shape and size in the direction from said foundation wall to said base plate.

7. The sperm sorting device as claimed in claim 1, wherein:
said base plate and said inner bordering wall of said channel unit and said foundation wall cooperatively define a solution-carrying space thereamong; and
said channel unit further has an inlet extending through said base plate and being in spatial communication with said solution-carrying space.

8. The sperm sorting device as claimed in claim 1, wherein said channel unit further has a recess extending from said second surface of said base plate towards said first surface.

9. The sperm sorting device as claimed in claim 1, wherein said foundation unit and said channel unit are made of biocompatible UV-curing resin.
